(19) United States
(12) Patent Application Publication
(10) Pub. No.: US 2021/0179986 A1
(43) Pub. Date: Jun. 17, 2021

(54) SYSTEM AND METHOD OF GROWING ALGAE USING GEOTHERMAL GAS

(71) Applicant: Algaennovation Ltd., Rosh Pinna (IL)

(72) Inventors: Ohad BASHAN, Sde Varburg (IL); Isaac BERZIN, Jerusalem (IL); Oded BASHAN, Rosh Pina (IL)

(73) Assignee: Algaennovation Ltd., Rosh Pinna (IL)

(21) Appl. No.: 16/761,665

(22) PCT Filed: Nov. 5, 2018
(86) PCT No.: PCT/IL2018/051177

§ 371 Date: May 5, 2020

Related U.S. Application Data
(60) Provisional application No. 62/581,789, filed on Nov. 6, 2017.

(51) Int. Cl. C12M 1/00; C12M 1/34; C12M 1/36; C12N 1/12

(52) U.S. Cl. CPC C12M 21/02; C12M 41/26; C12N 1/12; C12M 41/48; C12M 41/34

(57) ABSTRACT

A method of growing algae in a cultivation container is disclosed. In some the method may include: circulating, via the cultivation container, in a closed loop, a first predetermined amount of gas mixture comprising a first type of gas and at least one second type of gas, the gas mixture may enter the container via one or more entrance spargers and exit via at least one exit pipe, the first type of gas may contain CO₂ at a known first amount; receiving signal indicative of the amount of CO₂, in the gas mixture; when the signal indicates that the amount of CO₂ drops below a first predetermined level, extracting a second predetermined amount of the gas mixture from the cultivation container: and adding an amount of the first type of gas to the gas mixture, equal to the second predetermined amount.

### TECHNICAL FIELD OF THE INVENTION

The invention generally relates to the field of algae cultivation in artificial conditions and more precisely using geothermal gas a source for CO₂ in algae cultivation.

### BACKGROUND OF THE INVENTION

Bio-reactors for algae cultivation in artificial conditions has become increasingly common for producing biomass. Algae (or microalgae)-water culture is introduced into a container comprising water and supplied (e.g., fed) with small bubbles of gas that includes CO₂ and air. The algae in the container is further exposed to illumination (either artificial illumination, or from sunlight). For the algae biomass to perform photosynthesize and grow, the CO₂ needs to be dissolved into the water surrounding the algae culture. In phototropic algae cultivation systems the major inputs (or macro-nutrients) for growth are light, CO₂, nutrients (such as Nitrogen, Phosphorus, etc.), and water with turbulent mixing in order to distribute those resources to individual algae cultivation cells.

Microalgae can be grown in many types of systems, such as flat panel photo-bio-reactors having efficient light capture and utilization, and high surface area-to-volume ratio. Light sources for algae growth can be any type of visible light in the range of about 400-700 nm wavelengths. Light emitting diodes (LEDs) have the capability of providing light of specific wavelengths, for example in the visible light (e.g., blue and/or red) wavelength range.

The CO₂/air mixture is form by mixing CO₂ reach gas (e.g., above 50 weight percent (wt. %) CO₂) and air. The mixture usually includes 10-30 wt. % CO₂. The gas mixture is introduced as small bubbles into the algae-water culture in the reactor. The portion of the gas not dissolved or consumed by the algae is released into the outside atmosphere. Only a very small portion of the CO₂ is consumed by the algae, therefore the majority of CO₂ is being released and wasted.

There are several resources for CO₂. CO₂ reach gas can be obtained by distillation from air or combustion of carbon based fuels such as methane. CO₂ reach gas may include gas that have more than 50 wt. % CO₂, for example, more than 80 wt. %, more than 85 wt. % and above. Another source for CO₂ may be partially purified geothermal gas. Geothermal gas contains, for example, about 74 wt. % CO₂ but is also highly contaminated with toxic gasses such as 23.32 wt. % H₂S and explosive gasses such as 0.81 wt. % hydrogen, and 0.34 wt. % methane. Therefore, geothermal gas cannot be mixed with air to avoid explosion. Furthermore, due to the toxicity of some of the gasses in the geothermal gas, introducing such toxic gasses into an algae culture may have undesired effect on the growth of algae. Even relatively purified geothermal gas still contains 0.79 wt. % H₂S and 2.16 wt. % hydrogen, which makes it problematic to be used as a provider of CO₂ for algae cultivation.

Accordingly, in order to use geothermal gas in algae cultivation a special system and method is required.

### SUMMARY OF THE INVENTION

Some aspects of the invention may be related to a method of growing algae in a cultivation container. In some embodiments, the method may include: circulating, via the cultivation container, in a closed loop, a first predetermined amount of gas mixture comprising a first type of gas and at least one second type of gas, the gas mixture enters the container via one or more entrance spargers and exit via at least one exit pipe, wherein the first type of gas contains CO₂ at a known first amount; receiving signal indicative of the amount of CO₂, in the gas mixture; when the signal indicates that the amount of CO₂ drops below a first predetermined level, extracting a second predetermined amount of the gas mixture from the cultivation container: and adding an amount of the first type of gas to the gas mixture, equal to the second predetermined amount.

In some embodiments, the first type of gas may further include a toxic gas at a known second amount. In some embodiments, the first type of gas may be a geothermal gas. In some embodiments, the first known amount may be at least 9 weight % CO₂. In some embodiments, the second type of gas may include N₂. In some embodiments, the method may further include receiving signal indicative of the amount of O₂ in the circulating gas mixture; and replacing the circulating gas mixture with a new first predetermined amount of the gas mixture when the amount of O₂ raised above a second predetermined level.

Some aspects of the invention may be related to an algae cultivation system, the system may include: a cultivation container having one or more entrance spargers for introducing a gas mixture into the cultivation container and an exit pipe for releasing the gas mixture from cultivation container; a circulating system for circulating the gas mixture in closed loop form the exit pipe back to the one or more entrance spargers; at least one sensor for detecting changes in an amount of CO₂ in the gas mixture and a first gas supply system for supplying a first type of gas. In some embodiments, the first type of gas may include CO₂ at a known first amount. In some embodiments, the system may further include: a second gas supply system for supplying a second type of gas; and a controller configured to: control the first gas supply system and the second gas supply system to supply to the circulating system a first predetermined amount of gas mixture comprising the first type of gas and second type of gas; receive from the sensor a signal indicative of the amount of CO₂ in the gas mixture; and when the signal indicates that the amount of CO₂ drops below a first predetermined level, open a valve to extract a second predetermined amount of the gas mixture from the cultivation container; and control the first gas supply system to supply to the circulating system the first type of gas in an amount equal to the second predetermined amount.

In some embodiments, each of the first and the second gas supply systems may include a valve controlled by the controller. In some embodiments, the system may further include a third gas supply system for providing substantially pure CO₂. In some embodiments, the at least one sensor is one of: pH sensor located at the cultivation container and CO₂ sensor located in a circulation pipe included in the circulation system.

In some embodiments, the algae cultivation system may further include an O₂ sensor for detecting changes in an amount of O₂ in the circulating gas mixture. In some embodiments, the controller may further be configured to: receive a signal indicative of the amount of O₂ in the circulating gas mixture; when the amount of O₂ raised above a second predetermined level, control a relive valve included in the circulation system to release the circulating gas mixture; and control the first gas supply system and the second gas supply system to supply to the circulating system the first predetermined amount of gas mixture comprising the first type of gas and second type of gas.

In some embodiments, the first type of gas further may include a toxic gas at a known second amount. In some embodiments, the first type of gas may be a geothermal gas. In some embodiments, the first known amount may be at least 9 weight % of CO₂. In some embodiments, the second type of gas contains N₂.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is an illustration of an algae cultivation system according to some embodiments of the invention; and

FIG. 2 is a flowchart of a method of growing algae in a cultivation container according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

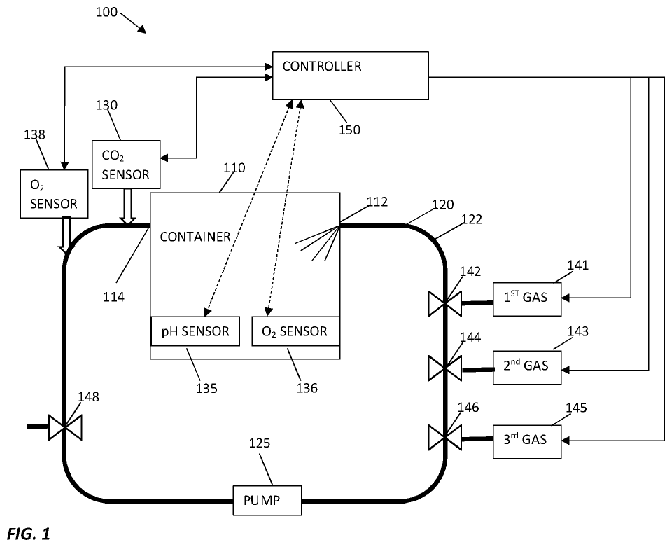

Reference is now made to FIG. 1, which schematically illustrates an algae cultivation system 100, according to some embodiments of the invention. System 100 may include a cultivation container 110 having one or more entrance spargers 112 for introducing gas into an algae culture and water located inside cultivation container 110 and an exit pipe 114 for releasing gas from cultivation container 110. System 100 may further include a circulating system 120 that includes a pipe 122 and a pump 125 for circulating gas in closed loop form the exit pipe 114 back to the one or more entrance spargers 112. System 100 may further include, at least one sensor 130 or 135 for detecting signals indicative of the amount of a first type of gas (e.g., CO₂), and two or more gas supply systems 141, 143 and 145 for providing a first type of gas, a second type of gas and/or a third type of gas to circulation system 120. In some embodiments, the system may include a relief valve 148 for relieving gas from circulation system 120. In some embodiments, system 100 may further include one or more O₂ sensors 136 and 138 for detecting changes in an amount of O₂ in the circulating gas mixture.

In some embodiments, system 100 may further include a controller 150 configured to receive signals from sensor 130 and/or sensor 135 and control valves 142-146 to open and allow gas supply systems 141, 143 and 145 to supply one or more of the first, second and third types of gasses to circulation system 120.

In some embodiments, container 110 may be any algae cultivation container known in the art. For example, container 10 may include at least two panels (not illustrated) within a water filled cultivation container 110, the panels positioned along a first plane the first plane is perpendicular to the gravitational force. In some embodiments, a cultivation volume may be created between each pair of panels, whereby the cultivation volumes may be fluidly coupled to allow horizontal flow therebetween along the first plane.

In some embodiments, one or more entrance spargers 112 may each include a plurality of nozzles, to distribute gas (e.g., CO₂, geothermal gas and/or air mixture) into cultivation container 110. In some embodiments, the gas bubbles may have a diameter of no more than 5 mm, for example, 2 mm or 1 mm. One or more entrance spargers 112 may supply the gas mixture to the container at 0.5-3 litter/minute for every liter of algae culture. In some embodiments, the bubbles may be introduced at a flow capacity sufficient for cleaning the walls of container 110 from biofilm covering the walls. In some embodiments, exit pipe 114 may include any device for collecting or extracting gasses from containers. Circulating system 120 may include one or more pipes 122 and any number of required connectors, valves, faucets and the like that may be required to allow forming a closed structure (a loop) for circulating gas form exit pipe 114 back to one or more entrance spargers 112. Circulating system 120 may include a pump 125 or a compressor or blower for evacuating the gas from the upper portion of container 110.

CO₂ sensor 130 may be any sensor configured to detect CO₂. For example, sensor 130 may be an infrared gas sensor (NDIR), a chemical gas sensor and the like. Sensor 135 may be any sensor that may provide indication to the amount of CO₂ in the algae culture, for example, sensor 135 may include a pH sensor. As the amount of CO₂ dissolve in the algae culture decrease below a required level the pH level may raise above a harmful level, which depends from the type of algae grow in cultivation container 110 (e.g., pH 7.5). Therefore, the pH may serve as an indicator to the amount of CO₂ in the gas mixture.

O₂ sensors 136 and 138 may be any sensors that may provide indication to the amount of O₂ in the gas mixture. Sensor 136 may be located at circulating container 110 configured to measure the amount of O₂ dissolved in the algae culture. Sensor 138 may be any chemical sensor configured to measure the amount of O₂ in the circulating gas mixture and may be located in pipe 122.

First gas supply system 141 may be configured to supply a first type of gas, containing CO₂ at a known first amount, for example, a geothermal gas or a partially purified geothermal gas. First gas supply system 141 may be in fluid communication with a reservoir (e.g., a tank, a pipe, etc.) that includes the first type of gas. In some embodiments, first gas supply system 141 may include valve 142, controlled by controller 150 and configured to supply the first type of gas to pipe 122 at a predetermined amount. In some embodiments, the known first amount may be at least 9 wt. % or more of CO₂. In some embodiments, the first type of gas may further include a toxic gas at a known second amount. For example, a partially purified geothermal gas may include up to 0.79 wt. % H₂S and 2.16 wt. % hydrogen. H₂S and/or hydrogen may be toxic to the algae and a system and method according to embodiments of the invention may be configured to control the amount of provided toxic gases to be below the toxicity level, for example, by continuous circulating the partially purified geothermal gas to cause maximum consumption of CO₂ by the algae without providing additional toxic gases, thus increase the level of toxicity to above a harming level.

Second gas supply system 143 may be configured to supply a second type of gas, for example, a gas containing nitrogen, such as air, nitrogen and the like. In some embodiments, second gas supply system 143 may include valve 144, controlled by controller 150 and configured to supply the second type of gas, for example, at a predetermined amount.

Third gas supply system 145 may be configured to supply a third type of gas, for example, a substantially pure CO₂ (e.g., a gas containing at least 90% CO₂). In some embodiments, third gas supply system 144 may include valve 146, controlled by controller 150 and configured to supply the third type of gas, for example, at a predetermined amount.

In some embodiments, only one or two types of gasses may be supplied via the one or more gas supply systems 141, 143 and 145. In such embodiments, some of gas supply systems may not be active, or may be operated to provide the same gas or gasses as another supply system. For example, gas supply systems 141 and 143 may supply a first type of gas and system 145 may supply a second type of gas. In another example, supply system 145 may not be operated, while each of supply systems 141 and 143 may supply a different type of gas.

Controller 150 may be any computation platform that is configured to perform instructions to control various components in system 100. Controller 150 may include a processor and a memory to store thereon instructions according to embodiments of the invention. Controller 150 may be configured to: control the first gas supply system to supply to the circulating pipe a first type of gas at a predetermined amount. For example, the controller may be configured to control supply system 141 to supply partially purified geothermal to pipe 120. and control second gas supply system 143 to supply to circulating pipe 120 the second type of gas for example, air or nitrogen. The first type of gas and the second type of gas may be supplied, to pipe 120, to form a first predetermined amount of gas mixture containing the first type of gas and second type of gas. The relative amounts of the first type of gas and the second type of gas, in the gas mixture, may also be predetermined, for example, based on the type of the algae growing in cultivation container 110. In some embodiments, the gas mixture may include (after mixing) at least 5 wt. % CO₂ The gas mixture may continually be circulated via container 110, by pump 125. During the circulation at least a portion of the small gas bubbles may dissolve into the water in container 110 and the CO₂ may be consumed by the algae. Therefore, in time the amount of the CO₂ may be reduced.

Controller 150 may further receive from sensor 130 and/or sensor 135 signal indicative to the amount of the CO₂ in the gas mixture. Controller 150 may continuously monitor the level of the CO₂ as the gas mixture being circulated via container 110. In some embodiments, when the amount of the CO₂ drops below a first predetermined level, controller 150 may open valve 148 to extract a second predetermined amount of the gas mixture from the cultivation container. Controller 150 may than control first gas system 141 to supply the first type of gas in an amount equal to the second predetermined amount. For example, when the a pH signal raised above 7.5, indicating that the amount of CO₂ is too low, controller 150 may control first gas system 141 to supply geothermal gas having 50% CO₂ to pipe 122. Alternatively, controller 150 may control third gas supply system 145 to supply CO₂ instead of geothermal gas.

In some embodiments, supplying controlled amounts of geothermal gas, may not harm the algae culture inside container 110 and may eliminate the risk of explosion.

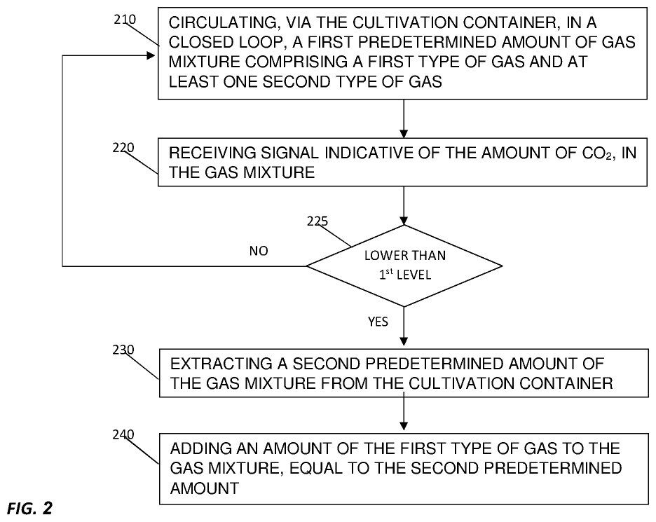

Reference is now made to FIG. 2 which is a flowchart of a method of growing algae in a cultivation container using, for example, geothermic gas, according to some embodiments of the invention. In box 210, a first predetermined amount of gas mixture comprising a first type of gas and at least one second type of gas, may be circulated, via a container (e.g., container 110), in a closed loop. In some embodiments, the gas mixture may enter the container via one or more entrance spargers (e.g., spargers 112) and exit via at least one exit pipe (e.g., exit pipe 114). For example, a mixture including a first type of gas containing known amount of CO₂ (e.g., geothermal gas containing at least 9 wt. % CO₂) and air may be circulated via a container (e.g., a bio-reactor) holding algae (e.g., micro-algae) and water. The entrance spargers may spray the gas mixture to form small bubbles having a diameter of no more than 5 mm, for example, 1 mm. In some embodiments, the first predetermined amount of gas mixture may be circulated continuously to allow consumption of the CO₂ from at least the first type of gas. In some embodiments, the first type of gas may include a toxic gas at a known second amount, for example, H₂S or hydrogen included in the geothermal gas. In some embodiments, the continuous circulation may allow the algae to consume the CO₂ from the geothermal gas before an additional amount of geothermal gas containing toxic gas(es) has to be added.

In some embodiments, the second type of gas may include gas containing N₂, such as, air. The second type of gas may provide the required bubbling and circulation for the algae culture, while the first type of gas may provide the nutrition, in the form of CO₂. In some embodiments, the ratio between the first type of gas and second type of gas in the gas mixture may be predetermined, for example, based on the type of algae. For example, the gas mixture may include at least 5% CO₂. The first predetermined amount may be determined to provide sufficient CO₂ and sufficient mixing and turbulence of the algae culture in container 110.

In box 220, signal indicative of the amount of CO₂, in the gas mixture may be received. For example, sensor 130 may continually measure the amount of CO₂ in pipe 122 and a controller such as controller 150 may monitor the measured amount. Additionally or alternatively, sensor 135 may monitor the pH level in container 110, the pH level may be indicative to the amount of CO₂ in the algae culture and the gas mixture.

If the signal indicative of the amount of CO₂ shows that the amount dropped below a predetermined level (box 225—YES), then in box 230, a second predetermined amount of the gas mixture from the cultivation container may be extracted. In some embodiments, controller 150 may control valve 148 to open and extract from circulation system 120, the second predetermined amount of gas mixture. The extracted gas mixture may have less relative amount of CO₂ than the gas mixture initially supplied in box 210. For example, 0.5 litters of circulated gas mixture per liter culture may be extracted when the pH of the algae culture reaches 7.5.

Following the extraction (e.g., after controller 150 closed valve 148) an amount of the first type of gas may be added to the gas mixture, in box 240. The added amount may be equal to the second predetermined amount. For example, controller 150 may control first gas system 141 to add 0.5 liters of geothermal gas per liter culture to circulation system 120.

In some embodiments, the process may continue until the amount of O₂ in the gas mixture and/or the algae capture reached a second predetermined level, for example, 35 wt. %. During the photosynthesis process the algae consumes the CO₂ and produces O₂ which above a certain amount prevents further grow of the algae. In some embodiments, the method may include replacing the circulating gas mixture with a new first predetermined amount of the gas mixture when the amount of O₂ raised above a second predetermined level. For example, controller 150 may control relive valve 148 to release the circulating gas mixture entirely. In this release all toxic gases accumulated in the circulating gas mixture are released as well. Following the release controller 150 may control first gas supply system 141 and the second gas supply system 143 to supply to the circulating system the first predetermined amount of gas mixture comprising the first type of gas and second type of gas, thus repeating the step in box 210.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method of growing algae in a cultivation container, the method comprising:
circulating, via the cultivation container, in a closed loop, a first predetermined amount of gas mixture comprising a first type of gas and at least one second type of gas, the gas mixture enters the container via one or more entrance spargers and exit via at least one exit pipe,
wherein the first type of gas contains CO₂ at a known first amount;
receiving signal indicative of the amount of CO₂, in the gas mixture;
when the signal indicates that the amount of CO₂ drops below a first predetermined level, extracting a second predetermined amount of the gas mixture from the cultivation container: and
adding an amount of the first type of gas to the gas mixture, equal to the second predetermined amount.

2. The method of claim 1, wherein the first type of gas further comprises a toxic gas at a known second amount.

3. The method of claim 2, wherein the first type of gas is a geothermal gas.

4. The method of claim 1, wherein the first known amount is at least 9 weight % CO₂.

5. The method of claim 1, wherein the second type of gas contains N₂.

6. The method of claim 1, further comprising:
receiving signal indicative of the amount of O₂ in the circulating gas mixture; and
replacing the circulating gas mixture with a new first predetermined amount of the gas mixture when the amount of O₂ raised above a second predetermined level.

7. An algae cultivation system, comprising:
a cultivation container having one or more entrance spargers for introducing a gas mixture into the cultivation container and an exit pipe for releasing the gas mixture from cultivation container;
a circulating system for circulating the gas mixture in closed loop form the exit pipe back to the one or more entrance spargers;
at least one sensor for detecting changes in an amount of CO₂ in the gas mixture;
a first gas supply system for supplying a first type of gas,
wherein the first type of gas contains CO₂ at a known first amount;
a second gas supply system for supplying a second type of gas; and
a controller configured to:
control the first gas supply system and the second gas supply system to supply to the circulating system a first predetermined amount of gas mixture comprising the first type of gas and second type of gas;
receive from the sensor a signal indicative of the amount of CO₂ in the gas mixture; and
when the signal indicates that the amount of CO₂ drops below a first predetermined level, open a valve to extract a second predetermined amount of the gas mixture from the cultivation container; and
control the first gas supply system to supply to the circulating system the first type of gas in an amount equal to the second predetermined amount.

8. The algae cultivation system of claim 7, wherein each of the first and the second gas supply systems comprises a valve controlled by the controller.

9. The algae cultivation system of claim 7, further comprising a third gas supply system for providing substantially pure CO₂.

10. The algae cultivation system of claim 7, wherein the at least one sensor is one of: pH sensor located at the cultivation container and CO₂ sensor located in a circulation pipe included in the circulation system.

11. The algae cultivation system of claim 7, further comprising:
an O₂ sensor for detecting changes in an amount of O₂ in the circulating gas mixture; and wherein the controller further configured to:
receive a signal indicative of the amount of O₂ in the circulating gas mixture;
when the amount of O₂ raised above a second predetermined level, control a relive valve included in the circulation system to release the circulating gas mixture; and
control the first gas supply system and the second gas supply system to supply to the circulating system the first predetermined amount of gas mixture comprising the first type of gas and second type of gas.

12. The algae cultivation system of claim 7, wherein the first type of gas further comprises a toxic gas at a known second amount.

13. The algae cultivation system of claim 12, wherein the first type of gas is a geothermal gas.

14. The algae cultivation system of claim 7, wherein the first known amount is at least 9 weight % of CO₂.

15. The algae cultivation system of claim 7, wherein the second type of gas contains N₂.
